(19) United States
(12) Patent Application Publication
(10) Pub. No.: US 2006/0020239 A1
(43) Pub. Date: Jan. 26, 2006

(54) Cerebral spinal fluid flow sensing device

(76) Inventors: Mark A. Geiger, Venture, CA (US); Lori C. Speckman, Ventura, CA (US)

Correspondence Address: SHUMAKER & SIEFFERT, P. A., 8425 SEASONS PARKWAY, SUITE 105, ST. PAUL, MN, 55125, US

(21) Appl. No.: 10/976,163

(22) Filed: Oct. 28, 2004

Related U.S. Application Data
(60) Provisional application No. 60/589,350, filed on Jul. 20, 2004.

(51) Int. Cl. A61M 5/00

(52) U.S. Cl. 604/9

(57) ABSTRACT

The disclosure is directed to an implantable device for sensing CSF flow through an implantable ventricular shunt. The sensing device is implanted with the CSF shunt, and includes a flow sensor to sense flow rate or shunt blockage. The sensing device is either placed within or adjacent to the fluid path through the shunt. The sensing device transmits the sensed flow rate to an external monitoring device by wireless telemetry. The sensing device may be integrally formed as part of the shunt, or clamped onto a portion of the shunt, in which case the sensing device may be resusable. An external monitor receives the transmitted flow signal and presents information based on the flow signal. The sensing device may be inductively powered or include its own power supply.

This application claims the benefit of U.S. provisional application No. 60/589,350, filed Jul. 20, 2004, the entire content of which is incorporated herein by reference.

### FIELD OF THE INVENTION

The invention relates to medical devices and, more particularly, devices for draining cerebrospinal fluid (CSF).

### BACKGROUND

Hydrocephalus is an excess accumulation of cerebrospinal fluid (CSF) in the ventricles of the brain. This fluid, which protects, nourishes and cleanses the brain and spinal cord, is manufactured daily in the ventricles. Accumulation occurs when the fluid cannot flow freely throughout the ventricles and the central nervous system due to various forms of blockage. Hydrocephalus can result from genetic conditions or trauma to the brain.

There are a number of accepted treatments available for hydrocephalus, most of which involve the surgical implantation of a ventricular shunt. A shunt diverts CSF from the ventricles to the peritoneal cavity or the cardiovascular system. The shunt may include a valve that controls the rate of flow of CSF through a catheter of the shunt. In some cases, the valve may be controllable to adjust the flow rate.

Some CSF shunt valves include a telemetric CSF flow or pressure sensor. U.S. Pat. No. 6,533,733 to Ericson et al. describes an implantable device for sensing intracranial pressure (ICP) and CSF flow, and transmitting sensed information to an external device by wireless telemetry. Zumkehr et al. describe an implantable telemetric CSF flow sensor in “Project: Packaging and Industrialization of an Implantable Flow Sensor for Neurological Applications,” CCP Center for Computational Physics, 2003. U.S. Pat. No. 4,519,401 to Ko et al. describes a battery-powered implantable ICP monitor with low power pressure sensing circuitry and wireless telemetry. U.S. Pat. No. 6,113,553 to Chubbuck describes an implantable, inductively powered ICP sensor providing wireless telemetry. U.S. Pat. No. 6,248,080 to Miesel et al. describes an implantable, battery-powered ICP sensor with wireless telemetry.

Table 1 below lists documents that disclose implantable telemetric CSF flow or ICP monitors.

TABLE 1

| Patent | | |
|---|---|---|
| Number | Inventors | Title |
| 6,113,553 | Chubbuck | Telemetric intracranial pressure monitoring |
| | | system |
| 6,533,733 | Ericson | Implantable device for in-vivo intracranial and |
| | et al. | cerebrospinal fluid pressure monitoring |
| 6,248,080 | Miesel | Intracranial monitoring and therapy delivery |
| | et al. | control device, system and method |
| 4,519,401 | Ko et al. | Pressure telemetry implant |
| Not | Zumkehr | Project: Packaging and Industrialization of an |
| applicable | et al. | Implantable Flow Sensor for Neurological |
| | | Applications |

All documents listed in Table I above are hereby incorporated by reference herein in their respective entireties. As those of ordinary skill in the art will appreciate readily upon reading the Summary, Detailed Description and Claims set forth below, many of the devices and methods disclosed in the patents of Table 1 may be modified advantageously by using the techniques of the present invention.

### SUMMARY OF THE INVENTION

In general, the invention is directed to an implantable device for sensing CSF flow through an implantable ventricular shunt. The sensing device is implanted with the CSF shunt, and includes a flow sensor to sense flow rate or shunt blockage. The sensing device transmits the sensed flow rate to an external monitoring device by wireless telemetry.

Various embodiments of the present invention provide solutions to one or more problems existing in the prior art with respect to prior art systems for CSF flow control in an implanted shunt. These problems include the insufficiency of ventricular size and ICP level, in some forms of hydrocephalus, as indicators of shunt performance. In particular, ventricular size or ICP level may not be a reliable indicator of the actual CSF flow rate through the shunt. The inability to accurately sense CSF flow rate can undermine the therapeutic efficacy of the shunt in treating hydrocephalus. In addition, without accurate CSF flow data, it may be difficult to identify an optimal valve pressure setting or changes in flow over time.

Various embodiments of the present invention are capable of solving at least one of the foregoing problems. When embodied in a system or method for monitoring CSF flow through an implantable ventricular shunt, the invention includes features that facilitate more accurate sensing of CSF flow rate. In one embodiment, the invention provides a system for monitoring CSF flow within a shunt. The system includes an implantable flow sensor configured to sense flow within the shunt and generate a flow signal, and a telemetry unit to transmit the flow signal by wireless telemetry. The sensing device may be integrally formed as part of the shunt, or clamped onto a portion of the shunt, in which case the sensing device may be resusable. An external monitor receives the transmitted flow signal and presents information based on the flow signal. The sensing device may be inductively powered or include its own power supply. In some embodiments, the flow sensor includes an optical sensor, such as a laser Doppler sensor, to sense CSF flow within a transparent or translucent inline tube that couples the CSF flow valve to a ventricular catheter within the shunt.

In comparison to known techniques for monitoring and controlling CSF flow in an implanted shunt, various embodiments of the invention may provide one or more advantages. For example, the invention enables a care-giver to better evaluate the performance of the ventricular shunt, and take remedial action if necessary. For example, if the sensed CSF flow rate is too low, too high, or indicates a blockage, the care-giver may adjust a flow control valve or take other action to address the situation. In either case, the invention provides a caregiver with the ability to determine and report shunt performance, and evaluate CSF flow data that may be helpful in identifying an optimal valve pressure setting, flow changes over time, or an obstruction in the system. Hence, an implantable CSF flow sensing device as described herein may aid the care-giver in ensuring proper shunt performance and adjustment. In addition, in some embodiments, the flow sensing device may be reusable upon replacement of all or a portion of an implanted shunt within a patient.

The above summary of the present invention is not intended to describe each embodiment or every embodiment of the present invention or each and every feature of the invention. Advantages and attainments, together with a more complete understanding of the invention, will become apparent and appreciated by referring to the following detailed description and claims taken in conjunction with the accompanying drawings.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating a CSF flow monitoring system having an implantable ventricular shunt and a CSF flow sensor in conjunction with a patient.

FIG. 2 is an enlarged side cross-sectional view of an inline CSF flow sensor.

FIG. 3 is a cross-sectional view of a CSF flow sensor taken along line 1-1′ of FIG. 2.

FIG. 4 is a cross-sectional view of an alternative inline CSF flow sensor.

FIG. 5 is a cross-sectional view of the flow sensor of FIG. 4 in an open configuration.

FIG. 6 is a block diagram illustrating a CSF flow sensor.

FIG. 7 is a block diagram illustrating an external monitor for receiving flow information from an implantable CSF flow sensor.

FIG. 8 is a flow diagram illustrating a method for monitoring CSF flow.

### DESCRIPTION OF THE PREFERRED EMBODIMENTS

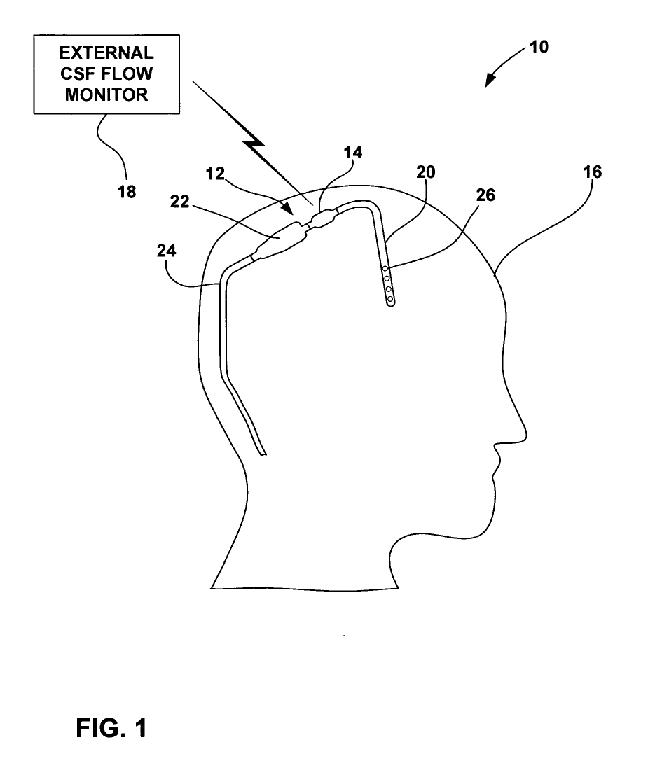

FIG. 1 is a schematic diagram illustrating a CSF flow monitoring system 10 having an implantable ventricular shunt 12 and a CSF flow sensor 14 in conjunction with a patient 16. CSF flow sensor 14 transmits CSF flow information to an external CSF flow monitor 18 via wireless telemetry. As shown in FIG. 1, shunt 12 is implanted within the cranium of patient 16, and includes a ventricular catheter 20, a CSF flow control valve 22 and a drainage catheter 24. Ventricular catheter 20, flow control valve 22 and drainage catheter 24 define a CSF flow path within shunt 12.

Ventricular catheter 20 may be bent and angled downward to extend into a brain ventricle. Alternatively, ventricular catheter 20 may be substantially straight, but couple to an angled coupling joint extending from control valve 22. Ventricular catheter 20 may define a plurality of holes 26 to receive CSF from a brain ventricle. Shunt 12 may be, for example, a ventriculoperitoneal shunt, a ventriculoatrial shunt, or a lumboperitoneal shunt.

CSF flow sensor 14 is constructed as an inline component that couples between one end of ventricular catheter 20 and one end of flow control valve 22. Hence, CSF flowing from ventricular catheter 20 to flow control valve 22 flows through CSF flow sensor 14. CSF flow sensor 14 includes a sensor to sense flow, and a wireless telemetry interface to transmit information based on the sensed flow rate to external CSF flow monitor 16. External CSF flow monitor 18 may provide further processing, analysis and presentation of the flow rate information for a care-giver.

Using the CSF flow information generated by CSF flow sensor 14, external monitor 18 may compute instantaneous flow rate, average flow rate, and generate trend data over a period of time. With the aid of this information, a care-giver may determine a more appropriate setting for valve 22 associated with shunt 12. CSF flow sensor 14 may be inductively powered, i.e., by an external inductive telemetric power source, or may include its own power source, such as a battery.

CSF flow sensor 14 and external monitor 18 provide a caregiver with the ability to determine and report shunt performance, and evaluate CSF flow data that may be helpful in identifying an optimal valve pressure setting, flow changes over time, or an obstruction in shunt. CSF flow sensor 14 may be useful with various CSF shunt and valve systems, such as those manufactured by Medtronic, Inc. of Minneapolis, Minn. Examples include the PS Medical® Delta®, Strata®, and CSF-Flow shunt and valve component systems commercially available from Medtronic.

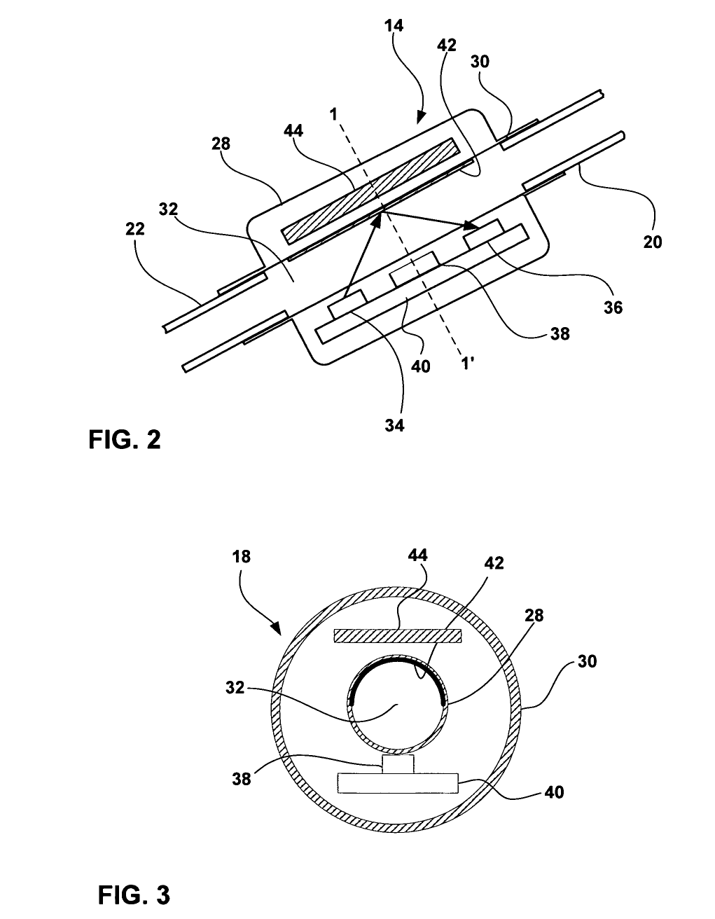

FIG. 2 is an enlarged side cross-sectional view of inline CSF flow sensor 14 of FIG. 1. FIG. 3 is an enlarged cross-sectional view of inline CSF flow sensor 14 taken along line 1-1′ of FIG. 2. As shown in FIG. 2, CSF flow sensor 14 is constructed for inline coupling between ventricular catheter 20 and CSF flow control valve 22 such that CSF flows from the ventricular catheter, through the CSF flow sensor, and to the flow control valve. To that end, CSF flow sensor 14 includes a housing 28 with an integrally formed tube 30 defining an inner lumen 32 to accommodate CSF flow.

Opposite ends of tube 30 receive and are coupled to ventricular catheter 20 and control valve 22, e.g., by crimping, adhesive bonding, ultrasonic bonding, or the like. In particular, an input of tube 30 is coupled to an output of ventricular catheter 20, and an output of tube 30 is coupled to an input of control valve 22. An output of control valve 22 is then coupled to an input of drainage catheter 24. In some cases, a length of an existing ventricular catheter 20 may be cut to accommodate attachment of tube 30 and CSF flow sensor 14.

Hence, flow control valve 22 has an input in fluid communication with an output of the ventricular catheter 20 and an output in fluid communication with an input of the drainage catheter 24. However, CSF flow sensor 14 is placed between the input of flow control valve 22 and the output of ventricular catheter 20. In other embodiments, CSF flow sensor 14 may be placed on an opposite side of control valve 22, i.e., between the output of the flow control valve and the input of the drainage catheter.

By constructing CSF flow sensor 14 as an integrally formed inline sensor, it may be incorporated in a variety of different shunt systems, and serve as a coupling member between ventricular catheter 20 and control valve 22. In some embodiments, housing 28 and tube 30 may be integrally molded from biocompatible plastic material, such as silicone or polyurethane.

In the example of FIG. 2, CSF flow sensor 14 includes an optical flow sensor. In other embodiments, CSF flow sensor 14 may incorporate alternative sensing devices such as electromagnetic flow meters, magnetic field flow meters, ultrasonic Doppler sensors, hot wire anemometers, thermal convection velocity sensors, or the like. As shown in FIG. 2, CSF flow sensor 14 includes an optical emitter 34, an optical receiver 36, and circuitry 38 for driving the optical emitter and receiver, as well as wireless telemetry circuitry. Emitter 34, receiver 36 and circuitry 38 may be mounted on a small circuit board 40. Emitter 34 may be a light emitting diode (LED) oriented to transmit light into inner lumen 32 of tube 30. Receiver 36 may be a photodiode sensitive to wavelengths of light transmitted by emitter 34, and may be positioned on the same side of tube 30 as emitter 34.

Optical emitter 34 transmits light through a translucent or transparent portion of the shunt tube 30 and into the flow field of the shunt 12, which may be integrally formed with the tube or retrofitted to the tube as part of the CSF flow sensor 14. In either case, optical receiver 36 receives reflected light and generates a CSF flow level signal based on the frequency, phase, or level of received light. CSF flow will tend to interrupt, attenuate, or shift the received light, and will provide an indication of CSF flow.

To permit transmission of light from emitter 34 into inner lumen 32, tube 30 may be constructed from a transparent or translucent material. Alternatively, tube 30 may include a transparent or translucent window oriented to permit transmission of light from emitter 34. Any of a variety of transparent or translucent materials may be used to construct tube 30. For example, tube 30 may be constructed from any of a variety of biocompatible glass or polymer compositions, such as transparent polyester, PTFE, or PVC. As an alternative, emitter 34 and receiver 36 may be designed to protrude, at least partially, into inner lumen 32 through fluid-sealed holes in tube 30. In this case, a transparent or translucent tube 30 is not necessary.

As an option, a reflector 42 may be mounted or coated onto an inner surface of tube 30. In this case, light produced by emitter 34 passes through CSF fluid within inner lumen 32 and reflects off of reflector 42 and toward receiver 36. Receiver 36 senses reflected light that passes through the CSF fluid twice, i.e., once from emitter 34 to reflector 42 and then again from the reflector to the receiver. Alternatively, receiver 36 may sense light reflected from the CSF fluid within inner lumen 32, rather than a reflector 42. As a further alternative, emitter 34 and receiver 36 may be mounted on opposing sides of tube 30 such that the receiver senses light that is transmitted from the emitter and through the CSF fluid within inner lumen 32.

In some embodiments, emitter 34 and receiver 36 may be constructed to form a laser Doppler sensor. In particular, emitter 34 may be constructed to illuminate the CSF flow within inner lumen 32 with a coherent beam of monochromatic light. Particles carried by the CSF, such as blood cells, reflect the light to produce back-scattered light that is Doppler-shifted relative to the transmitted light. Receiver 36 senses the back-scattered light, and generates an output signal for circuitry 38.

Circuitry 38 processes the output signal to produce a flow rate signal. For example, circuitry 38 may be configured to mix the output signal with an input signal used to drive emitter 34, and thereby generate a difference signal. The difference signal is based on a difference between the frequency of the light transmitted by emitter 34 and the Doppler-shifted frequency of the reflected light received by receiver 36. The magnitude of the difference signal indicates the flow rate of the CSF through the inner lumen 32 of tube 30, and hence through shunt 12. In particular, the difference signal is proportional to the rate at which particles carried by the CSF fluid travel through inner lumen 32.

With this information, circuitry 38 generates a CSF flow rate signal. In some embodiments, circuitry 38 may include additional processing circuitry to evaluate the CSF flow rate signal relative to a desired maximum or minimum flow rate and possibly identify blockages. Alternatively, such processing may be performed within external CSF flow monitor 18 to reduce processing overhead and power consumption within CSF flow rate sensor 14. In some embodiments, external CSF flow monitor 18 may provide advisories in the event the CSF flow rate deviates from a desired range. In addition, external CSF monitor 18 may present recommendations for adjustments to flow control valve 22.

Circuitry 38 further includes telemetry circuitry that drives an inductive coil 44 to transmit a telemetry signal based on the computed flow rate. As will be described, inductive coil 44 also may be used to receive power from an external power source. In this case, circuitry 38 also may include inductive power generation circuitry to convert current induced in inductive coil 44 into operating power for the sensing and telemetry functions performed by CSF flow sensor 14. Hence, CSF flow sensor 14 may be telemetrically powered. As an alternative, in some embodiments, CSF flow sensor 14 may include a rechargeable or non-rechargeable battery. A rechargeable battery may be recharged using current induced in inductive coil 44 by an external power source.

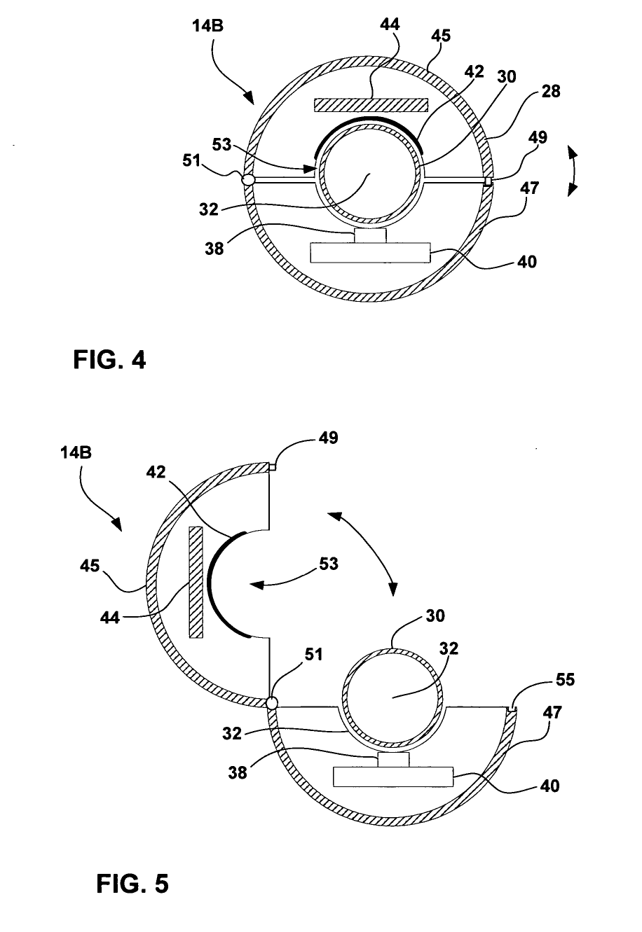

FIG. 4 is a cross-sectional view of an alternative CSF flow sensor 14B. In the example of FIGS. 2 and 3, CSF flow sensor 14 included an integrally formed housing 28 and tube 30 designed for inline coupling between ventricular catheter 20 and control valve 22. CSF flow sensor 14B generally corresponds to CSF flow sensor 14 of FIGS. 2 and 3. In the example of FIG. 4, however, CSF flow sensor 14B is designed to attached to a section of ventricular catheter 20 or a section of tubing between the ventricular catheter and control valve 22. In particular, CSF flow sensor 14 B of FIG. 4 is designed with a “clam-shell” configuration that permits it to be clamped onto an existing section of transparent or translucent tubing, and possibly detached for reuse, if desired.

FIG. 5 is a cross-sectional view of flow sensor 14B of FIG. 4 in an open configuration. As shown in FIGS. 4 and 5, flow sensor 14B includes a housing 28 with a first half 45 and a second half 47 that are coupled together with a snap-fit post 49 and a hinge 51. First half 45 contains coil 44 and optional reflector 42, while second half 47 contains circuit board 40 and circuitry 38. Flexible electrical cabling may extend between coil 44 and circuit board 40, e.g., along the hinge point defined by hinge 51.

First and second housing halves 45, 47 pivot about hinge 51 to permit the halves to be placed about a tube 30. Halves 45, 47 define an inner cavity 53 sized to accommodate tube 30. By closing halves 45, 47 about hinge 51 and engaging snap-fit post 49 with socket 55, flow sensor 14B can be clamped around tube 30. Cavity 53 is sized so that emitter 34 and receiver 36 (not shown in FIGS. 4 and 5) are placed sufficiently close to tube 30 to sense flow within inner lumen 32.

If shunt 12 is replaced, either partially or in its entirety, flow sensor 14B can be detached and then reapplied to the replacement shunt using the snap-fit arrangement. For example, snap-fit post 49 and socket 55 may be sized so that the post remains engaged once it is urged into the socket, but can be disengaged from the socket upon application of force to permit reuse. Although a clam-shell arrangement is depicted in FIGS. 4 and 5, other arrangements such as screw-tightened or friction fit C-clamps may be used, provided they do not cause an obstruction in tube 30. In each case, flow sensor 14B can be quickly added to an existing shunt 12 with transparent or translucent tubing, and reused with another shunt, as needed.

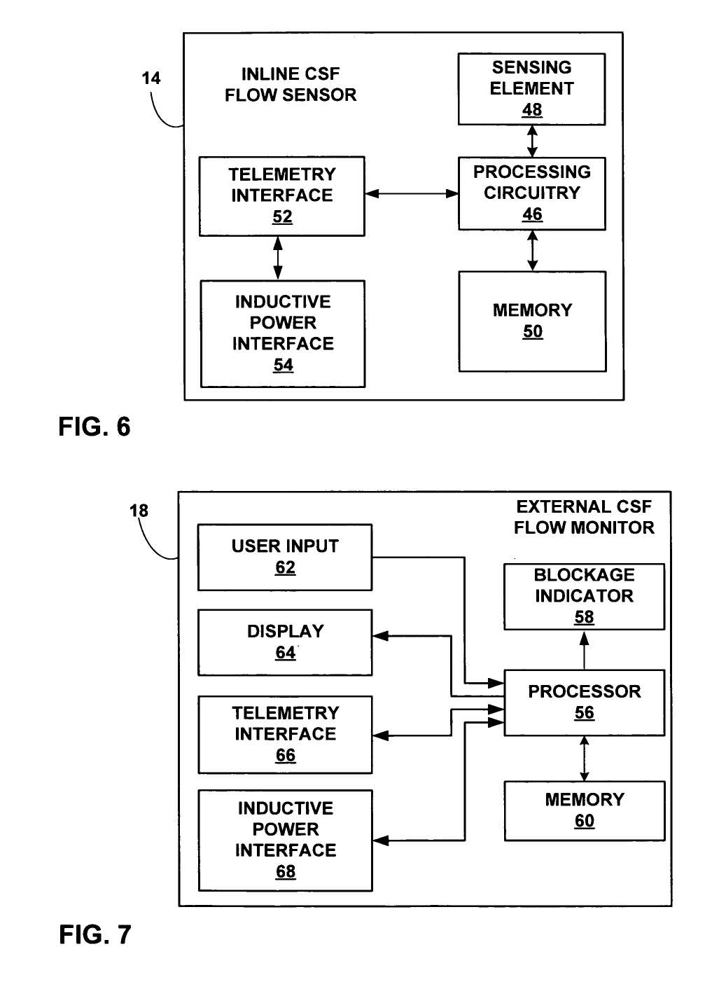

FIG. 6 is a block diagram illustrating a CSF flow sensor 14. As shown in FIG. 6, flow sensor 14 may include processing circuitry 46, sensing element 48, memory 50, telemetry interface 52 and inductive power interface 54. Sensing element 48 may include an emitter and receiver, as described with reference to FIGS. 2 and 3, for optical flow sensing. Processing circuitry 46 may form part of circuitry 38 shown in FIGS. 2 and 3. Processing circuitry 46 controls the operation of the various components of sensing element 48 and provides the processing resources to handle processing of output signals from the sensing element. Processing circuitry 46 also controls telemetry interface 52 to transmit signals to external CSF flow monitor 18.

Processing circuitry 46 may include one or more microprocessors, digital signal processors (DSPs), application-specific integrated circuits (ASICs), field-programmable gate arrays (FPGAs), or other equivalent processing circuitry. Memory 50 may include random access memory (RAM), read-only memory (ROM), electronically-erasable programmable ROM (EEPROM), flash memory, or the like, or a combination thereof. Memory 50 may store program instructions that are executed by processing circuitry 46 to perform some of the functions described herein. For example, memory 50 may store instructions for processing circuitry 46 to execute in support of control of telemetry interface 52 and control of, and processing of information obtained from, CSF flow sensor 14. Memory 50 may include separate memories for storage of instructions and archived CSF flow information.

Telemetry interface 52 includes an inductive coil for wireless communication as well as inductive generation of operating power for flow sensor 14. Telemetry interface 52 also may include appropriate modulation, filtering and amplification circuitry for transmission of signals under control of processor 46. Inductive power interface 54 converts alternating current (ac) induced in the inductive coil into operating power for processing circuitry 46, sensing element 48, memory 50, and telemetry interface 52. For example, inductive power interface 54 may include an ac/dc conversion circuit, such as a rectifier, that converts the ac current induced in the coil into dc operating power.

Inductive power interface 54 also may include a capacitor or other storage device to store a dc potential as a source of operating power. The capacitor may store energy temporarily to power flow sensor 14, e.g., only during the time that inductive coil actually receives energy external CSF flow monitor 18. Alternatively, a battery may be provided to power flow sensor 14 over an extended period of time. In some embodiments, inductive power interface 54 may generally correspond to similar circuitry described in U.S. Pat. No. 6,731,976 to Penn et al., the entire content of which is incorporated herein by reference.

Processing circuitry 46 filters, amplifies, and processes the ICP measurement signal, as necessary. Telemetry interface 52 then generates telemetry signals for wireless transmission to external monitor 18. Telemetry interface 52 includes appropriate amplifier, filtering and modulation circuitry to convert the ICP measurement signal into a telemetry signal.

FIG. 7 is a functional block diagram illustrating an external monitor 18 for receiving flow information from an implantable CSF flow sensor 14 and powering the implantable CSF flow sensor. External CSF flow monitor 18 provides an indication of CSF flow rate measured by implantable CSF flow sensor 14, e.g., on a display device or printout. In addition, external monitor 18 may be configured to apply additional processing to CSF flow information received from CSF flow sensor 14 to generate average, maximum, minimum and trend data. In some embodiments, external monitor 18 may generate recommendations for adjustments to control valve 22 to achieve an optimum flow rate. Also, external monitor 18 may invoke advisory levels at which a CSF flow measurement may trigger an alarm or other indicator for the attention of a care-giver. For example, external monitor 18 may alert a care-giver to the presence of a blockage or an undesirably high or low flow rate.

As shown in FIG. 7, external monitor 18 may include a processor 56, blockage indicator 58, memory 60, user input device 62, display 64, telemetry interface 66 and inductive power interface 68. Processor 56 controls the operation of the various components of external monitor 18. For example, processor 56 controls inductive power interface 68 and telemetry interface 66, and handles processing and storage of information obtained from implantable CSF sensor 14. Processor 56 may include one or more microprocessors, digital signal processors (DSPs), application-specific integrated circuits (ASICs), field-programmable gate arrays (FPGAs), or other equivalent logic circuitry.

Processor 56 also may accept input from user input device 62, e.g., to select different formats, or time or amplitude scales, for presentation of CSF flow information on display 64. Display 64 may include any of a variety of different displays, such as a liquid crystal display (LCD), plasma display, or cathode ray tube (CRT) display. In addition, processor 56 may archive CSF flow information within memory 60 for retrieval or transmission to other devices, such as remote monitors distributed within a network.

Memory 60 may include any magnetic, electronic, or optical media, such as random access memory (RAM), read-only memory (ROM), electronically-erasable programmable ROM (EEPROM), flash memory, or the like, or a combination thereof. Memory 60 may store program instructions that, when executed by processor 56, cause the processor to perform the functions ascribed to it herein. For example, memory 60 may store instructions for processor 56 to execute in support of control of wireless telemetry interface 66 and control of, and processing of information obtained from implantable CSF sensor 14. Memory 60 may include separate memories for storage of instructions and archived CSF flow or ICP information.

Telemetry interface 66 may include a wireless radio frequency (RF) receiver and suitable demodulation, amplification and filtering circuitry to permit reception of information transmitted by implanted CSF flow sensor 14. In some embodiments, CSF flow sensor 14 may be equipped for bidirectional communication, and may be responsive to commands transmitted via telemetry interface 66. In each case, telemetry interface 66 includes an antenna, which may take the form of an inductive coil placed adjacent the patient's head to ensure reliable telemetry. In particular, the inductive coil may be embedded in a wand-like instrument.

Inductive power interface 66 applies current to an inductive coil to support inductive transfer of electromagnetic energy to implanted CSF flow sensor 14. Typically, the same inductive coil associated with external monitor 18 will be used for both telemetry and inductive power transfer, as is the case with the inductive coil within CSF flow sensor 14. Telemetry interface 66 and inductive power interface 68 enable CSF flow sensor 14 to be operated passively. In other words, all of the power for operation of CSF flow sensor 14 is provided by external monitor 18. When inductive power interface 68 is activated, CSF flow sensor 14 senses CSF flow rate within shunt 12 and transmits flow rate information to external monitor 18 by wireless telemetry.

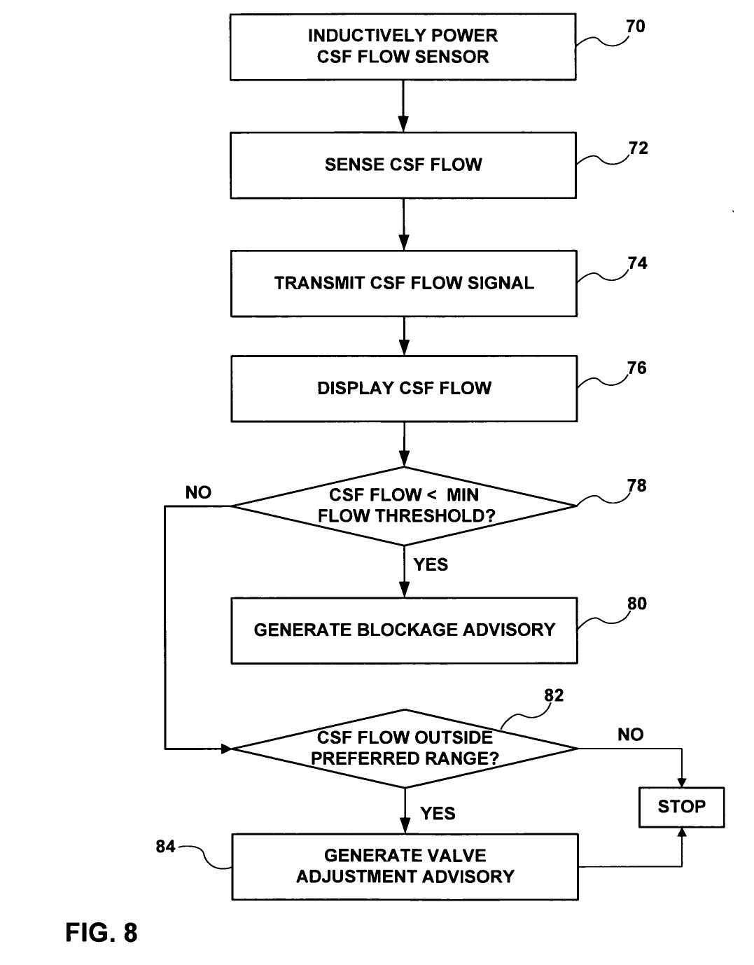

FIG. 8 is a flow diagram illustrating a method for monitoring CSF flow. The method may be implemented by implanted CSF flow sensor 14 and external monitor 18. As shown in FIG. 8, external monitor 18 inductively powers CSF flow sensor 14 (70). In response, CSF flow sensor 14 optically senses CSF flow (72) within shunt 12, and transmits a CSF flow signal (74) to external monitor 18 by wireless telemetry. External monitor 18 displays CSF flow information based on the CSF flow signal (76) for evaluation by a care-giver.

In addition, external monitor 18 may be configured to apply further processing and analysis to the CSF flow information. For example, external monitor 18 may compare the CSF flow rate to a minimum flow threshold (78). If the CSF flow rate is less than the minimum flow threshold, external monitor 18 generates a blockage advisory (80), which may be presented to the care-giver visually or audibly. For example, the blockage advisory may be presented visually via display 64 or a dedicated light, and audibly in the form of an audible tone or speech. In each case, the care-giver may take appropriate intervention steps to address the detected blockage.

If the CSF flow is not less than the minimum flow threshold (78), but does not fall within a desired operational range (82), external monitor 18 may generate a valve adjustment advisory (84) for the care-giver. In some embodiments, the valve adjustment advisory may simply indicate that a valve adjustment is necessary. In other embodiments, the valve adjustment advisory may specify the magnitude of the valve adjustment necessary to restore CSF flow to the desired range. In this manner, external monitor 18 may be helpful in determining an optimum valve setting.

In general, CSF flow sensor 14 provides a caregiver with the ability to determine and report shunt performance, and evaluate CSF flow data that may be helpful in identifying an optimal valve pressure setting, flow changes over time, or an obstruction in shunt 12. CSF flow sensor may take a variety of forms, as described herein, but preferably is constructed as a sensor that is either placed within or adjacent to the fluid flow path through shunt 12.

Again, CSF flow sensor 14 may incorporate an optical sensor, such as laser Doppler sensor, as described with reference to FIGS. 2-5. However, other types of sensors may be suitable, such as electromagnetic flow meters, pressure-based flow sensors, magnetic field flow sensors, ultrasonic Doppler flow sensors, thermal flow sensors, and other types of optical sensors. As one example, although a laser Doppler sensor has been described, an alternative type of optical sensor may simply rely on a level of reflected or transmitted light passing through the CSF flow. In this case, blood cells in the CSF serve to interrupt or attenuate transmitted light, such as infrared light. The resulting level of received light, attenuated by the CSF, indicates the rate of CSF flow.

The preceding specific embodiments are illustrative of the practice of the invention. It is to be understood, therefore, that other expedients known to those skilled in the art or disclosed herein may be employed without departing from the invention or the scope of the claims.

In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

Various embodiments of the invention have been described. Various modifications may be made without departing from the scope of the claims. These and other embodiments are within the scope of the following claims.

1: A cerebral spinal fluid (CSF) shunt system comprising:
a ventricular catheter;
a drainage catheter;
a flow control valve coupled between the ventricular catheter and the drainage catheter, wherein the ventricular catheter, the flow control valve and the drainage catheter define a CSF flow path; and
a CSF flow sensor positioned to sense flow within the CSF flow path, the CSF flow sensor having a tube in fluid communication with an output of the ventricular catheter and an input of the flow control valve, and an optical sensor that senses CSF flow within the tube.

2: The system of claim 1, wherein the optical sensor includes an optical emitter that emits light into the tube and an optical receiver that receives light from the tube, the optical sensor sensing CSF flow based on the received light, and wherein the tube is at least partially transmissive to the light emitted by the emitter.

3: The system of claim 2, further comprising a reflector mounted on a side of the tube opposite the emitter to reflect the light emitted by the emitter.

4: The system of claim 1, wherein the CSF flow sensor is a laser Doppler sensor, wherein the laser Doppler sensor includes an optical emitter that emits light into the CSF flow path, an optical receiver that receives light from the CSF flow path, and processing circuitry to sense CSF flow based on a difference in frequency between the light emitted by the optical emitter and light received by the optical receiver.

5. (canceled)

6: The system of claim 1, wherein the CSF flow sensor includes a wireless telemetry interface to transmit information based on the sensed CSF flow to an external monitor.

7: The system of claim 1, wherein the CSF flow sensor includes an inductive power interface to receive power from an external device.

8: The system of claim 1, further comprising an external monitor to receive CSF flow information transmitted by the CSF flow sensor.

9: The system of claim 8, wherein the external monitor includes an inductive power interface to power the CSF flow sensor.

10: A cerebral spinal fluid (CSF) shunt system comprising:
a ventricular catheter;
a drainage catheter;
a flow control valve coupled between the ventricular catheter and the drainage catheter, wherein the ventricular catheter, the flow control valve and the drainage catheter define a CSF flow path; and
a CSF flow sensor configured to attach to a portion of the CSF flow path to sense flow within the CSF flow path.

11: The system of claim 10, wherein the CSF flow sensor is an optical sensor, and the portion of the CSF flow path is at least partially transmissive to light.

12: The system of claim 11, wherein the CSF flow sensor is a laser Doppler sensor, wherein the laser Doppler sensor includes an optical emitter that emits light into the CSF flow path, an optical receiver that receives light from the CSF flow path, and processing circuitry to sense CSF flow based on a difference in frequency between the light emitted by the optical emitter and light received by the optical receiver.

13. (canceled)

14: The system of claim 10, wherein the CSF flow sensor has a clam-shell configuration defining first and second portions that are pivotable about a hinge point to clamp onto the flow path.

15: The system of claim 14, further comprising a snap-fit engagement to couple the first and second portions of the clam-shell configuration.

16: The system of claim 10, wherein the CSF flow sensor is attached to a portion of the ventricular catheter.

17: The system of claim 10, wherein the CSF flow sensor includes a wireless telemetry interface to transmit information based on the sensed CSF flow to an external monitor.

18: The system of claim 10, wherein the CSF flow sensor includes an inductive power interface to receive power from an external device.

19: The system of claim 10, further comprising an external monitor to receive CSF flow information transmitted by the CSF flow sensor.

20: The system of claim 19, wherein the external monitor includes an inductive power interface to power the CSF flow sensor.

21: A cerebral spinal fluid (CSF) flow sensor for sensing CSF flow within a ventricular shunt, the sensor comprising:
a sensor housing;
a tube within the sensor housing to receive CSF from a flow path within a CSF shunt;
an optical sensor, mounted within the housing, to sense CSF flow within the tube;
a wireless telemetry interface, mounted within the housing, to transmit information based on the sensed CSF flow to an external monitor; and
an inductive power interface, mounted within the housing, to receive power from an external device.

22: The sensor of claim 21, wherein the optical sensor includes an optical emitter that emits light into the tube and an optical receiver that receives light from the tube, the optical sensor sensing CSF flow based on the received light, and wherein the tube is at least partially transmissive to the light emitted by the emitter.

23: The sensor of claim 22, further comprising a reflector mounted on a side of the tube opposite the emitter to reflect the light emitted by the emitter.

24: The sensor of claim 21, wherein the optical sensor is a laser Doppler sensor, wherein the laser Doppler sensor includes an optical emitter that emits light into the CSF flow path, an optical receiver that receives light from the CSF flow path, and processing circuitry to sense CSF flow based on a difference in frequency between the light emitted by the optical emitter and light received by the optical receiver.

25. (canceled)

26: The sensor of claim 21, wherein the tube includes a first end for connection to an output of a ventricular catheter and a second end for connection to an input of a control valve.

27: A cerebral spinal fluid (CSF) flow sensor for sensing CSF flow within a ventricular shunt, the sensor comprising:
a sensor housing configured to attach to a portion of a CSF flow path within a CSF shunt;
a sensor, mounted within the housing, to sense flow of CSF within the flow path;
a wireless telemetry interface, mounted within the housing, to transmit information based on the sensed CSF flow to an external monitor; and
an inductive power interface, mounted within the housing, to receive power from an external device.

28: The sensor of claim 27, wherein the CSF flow sensor is an optical sensor, and the portion of the CSF flow path is at least partially transmissive to light.

29: The sensor of claim 28, wherein the optical sensor is a laser Doppler sensor, wherein the laser Doppler sensor includes an optical emitter that emits light into the CSF flow path, an optical receiver that receives light from the CSF flow path, and processing circuitry to sense CSF flow based on a difference in frequency between the light emitted by the optical emitter and light received by the optical receiver.

30. (canceled)

31: The sensor of claim 27, wherein the CSF flow sensor has a clam-shell configuration defining first and second portions that are pivotable about a hinge point to clamp onto the portion of the flow path.

32: A method for sensing CSF flow in a ventricular shunt system, the method comprising:
attaching a CSF flow sensor to a portion of a CSF flow path defined by a ventricular catheter, flow control valve and drainage catheter, wherein the portion of the flow path is substantially transmissive to light;
telemetrically powering the CSF flow sensor to optically sense CSF flowing through the flow path; and
receiving CSF flow information from the CSF flow sensor via wireless telemetry.

33: The method of claim 32, wherein the CSF flow sensor is a laser Doppler sensor, wherein the laser Doppler sensor includes an optical emitter that emits light into the CSF flow path, an optical receiver that receives light from the CSF flow path, and processing circuitry to sense CSF flow based on a difference in frequency between the light emitted by the optical emitter and light received by the optical receiver.

34. (canceled)

35: The method of claim 32, wherein the CSF flow sensor has a clam-shell configuration defining first and second portions that are pivotable about a hinge point, the method further comprising closing the first and second portions to clamp the clam-shell configuration about the portion of the flow path.

36-39. (canceled)
